(19) United States
(12) Patent Application Publication
(10) Pub. No.: US 2026/0174448 A1
(43) Pub. Date: Jun. 25, 2026

(54) THROMBECTOMY APPARATUS AND METHOD

(71) Applicant: Varian Medical Systems, Inc., Palo Alto, CA (US)

(72) Inventor: Francis Facchini, Hinsdale, IL (US)

(21) Appl. No.: 19/067,114

(22) Filed: Feb. 28, 2025

Related U.S. Application Data
(63) Continuation-in-part of application No. 18/999,414, filed on Dec. 23, 2024.

(51) Int. Cl. A61B 17/22; A61B 17/00; A61B 90/92; A61B 90/94

(52) U.S. Cl. CPC A61B 17/22; A61B 90/92; A61B 90/94; A61B 2017/00292; A61B 2017/00561; A61B 2017/22079

(57) ABSTRACT

A thrombectomy kit and method provides for aspirating a thrombus without use of a syringe that is precharged with a vacuum.

### CROSS-REFERENCE TO RELATED APPLICATIONS

This application is a continuation-in-part of U.S. application Ser. No. 18/999,414 filed Dec. 23, 2024, which is incorporated herein by reference in its entirety.

### TECHNICAL FIELD

These teachings relate generally to treating a patient's thrombus and more particularly to conducting a thrombectomy.

### BACKGROUND

A thrombus is a clot of blood formed within a blood vessel that remains attached to its place of origin (with a plurality of such clots being referred to as thrombi). A thrombus can be the cause of a stroke. A stroke occurs when the blood supply to part of the brain is interrupted or reduced, preventing brain tissue from getting oxygen and nutrients, which can cause brain cells to die. A stroke caused by a thrombus is a significant cause of death and disability. A thrombus can occur in any other part of the body including the heart, the liver, kidney, extremities including arms and legs, and so forth.

One approach to removing a thrombus using aspiration has the clinician withdraw the plunger of an aspiration syringe that is connected to a thrombectomy catheter via a closed valve to a significant extent. Doing so creates a vacuum in the syringe and hence precharges the syringe with a vacuum. The clinician then opens the aforementioned valve and the sudden presentation of that precharged vacuum imposes a sudden force on the thrombus to hopefully fully aspirate the clot.

Unfortunately, these prior art approaches may be inadequate, at least in some application settings, for effectively serving as intended due to such things as limitations with respect to the application of force, thrombus positioning, and the risk of clot fragmentation, which can lead to further embolization, vessel occlusion, and resulting subsequent complications. Current thrombectomy approaches also may not be sufficiently gentle on fragile blood vessels.

### BRIEF DESCRIPTION OF DRAWINGS

Various needs are at least partially met through provision of the thrombectomy kit, apparatus, and method described in the following detailed description, particularly when studied in conjunction with the drawings, wherein:

FIG. 1 comprises a block diagram as configured in accordance with various embodiments of these teachings;

FIG. 2 comprises a perspective/schematic diagram as configured in accordance with various embodiments of these teachings;

FIG. 3 comprises a flow diagram as configured in accordance with various embodiments of these teachings; and

FIG. 4 comprises a block diagram as configured in accordance with various embodiments of these teachings.

Elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present teachings. Also, common but well-understood elements that are useful or necessary in a commercially feasible embodiment are often not depicted in order to facilitate a less obstructed view of these various embodiments of the present teachings. Certain actions and/or steps may be described or depicted in a particular order of occurrence while those skilled in the art will understand that such specificity with respect to sequence is not actually required. The terms and expressions used herein have the ordinary technical meaning as is accorded to such terms and expressions by persons skilled in the technical field as set forth above except where different specific meanings have otherwise been set forth herein. The word “or” when used herein shall be interpreted as having a disjunctive construction rather than a conjunctive construction unless otherwise specifically indicated.

### DETAILED DESCRIPTION

Generally speaking, pursuant to these various embodiments, a thrombectomy kit can comprise a thrombectomy catheter having a distal end, at least one syringe, a valve configured to operably couple between the syringe and the distal end of the thrombectomy catheter, and thrombectomy instructions that instruct a user to use the components of the thrombectomy kit to remove a thrombus without a precharged vacuum in the syringe.

By one approach, the valve and the syringe can include indicators to visually identify which is to be used first when initiating removal of a thrombus. By one approach, the aforementioned indicators comprise different colors. By another approach, in lieu of the foregoing or in combination therewith, the indicators comprise graphics. And by another approach, again in lieu of the foregoing or in combination therewith, the indicators can comprise alphanumeric characters. Such indicators can serve to help the clinician inappropriately precharge the syringe with a vacuum before opening the valve.

By one approach, the thrombectomy kit can further include mechanical linkage that couples the syringe to the valve, such that withdrawing a plunger from the syringe simultaneously opens the valve to thereby prevent precharging the syringe with a vacuum.

By one approach, the aforementioned thrombectomy instructions can include illustrations that positively illustrate using the components of the thrombectomy kit without a precharged vacuum. In lieu of the foregoing or in combination therewith, the thrombectomy instructions can include illustrations that negatively illustrate using the components of the thrombectomy kit with a precharged vacuum.

By one approach, the thrombectomy kit includes a plurality of syringes as well as a manifold that is configured to operably couple the plurality of syringes to the thrombectomy catheter via the valve.

By one approach, the thrombectomy kit can further include at least one mechanical thrombectomy component that is configured to be disposed through a patient's blood-carrying vessel to a thrombus that is to be removed from the patient. For example, by one approach the at least one mechanical thrombectomy component can comprise a clot retriever (such as, but not limited to, a clot entrapment cage).

The instructions provided in the thrombectomy kit can provide instructions for proper use of the components in the kit. Generally speaking, those instructions can convey:
disposing a thrombectomy catheter within a patient having at least one thrombus to be removed from the patient such that an inserted end of the thrombectomy catheter is located proximal to the at least one thrombus;
connecting a syringe in fluid communication to the thrombectomy catheter via a closed valve;
opening the closed valve without first having precharged the syringe with a vacuum;
applying a gradually increasing pressure to the syringe (by withdrawing the corresponding plunger) while monitoring a resistance to applying the pressure to the syringe and thrombectomy catheter;
modifying one or more pressure-related parameters related to an application of pressure to the syringe based on a result of the monitoring; and
aspirating at least a portion of the at least one thrombus using the application of pressure to the syringe as customized based on the monitoring.

By one approach, the aforementioned monitoring is done via the senses of the clinician and their interpretation of what they are sensing based on their thrombectomy experience. The aforementioned one or more pressure-related parameters can comprise, for example, at least one of a speed and/or force of applying pressure, a duration of applying pressure, and time intervals between each application of pressure.

So configured, neither the thrombus nor the vessel are automatically subjected to sudden forces during the thrombectomy procedure, because the syringe is not precharged with a vacuum to present such forces when the valve is suddenly initially opened. Instead, withdrawal of the thrombus occurs at a rate and under circumstances that, at all times, are f under the control of the clinician.

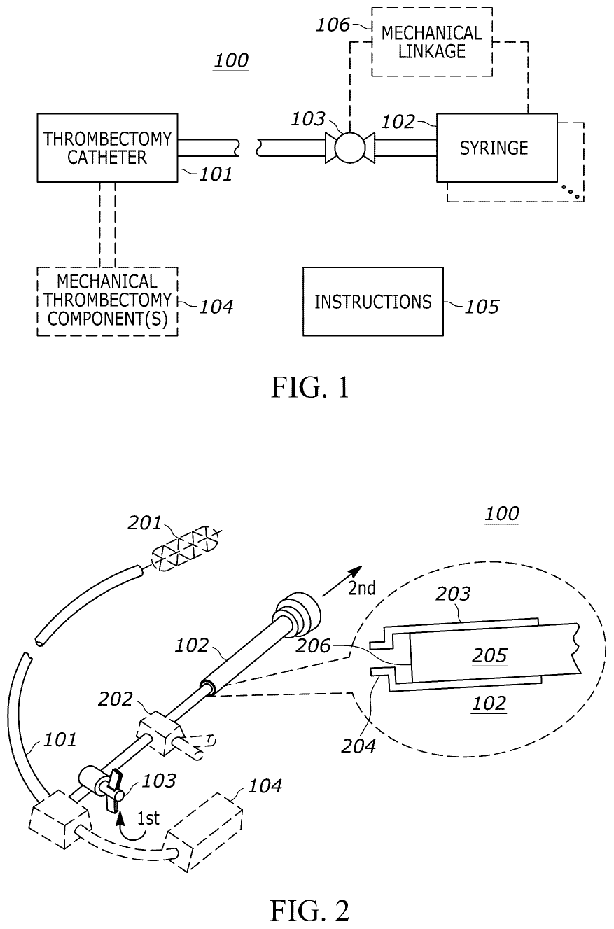

FIGS. 1 and 2 present an illustrative example of a thrombectomy apparatus kit 100 that accords with the present teachings.

The thrombectomy apparatus kit 100 includes a syringe 102. As shown in more detail in FIG. 2, this syringe 102 includes a barrel 203 having a tip 204. A plunger 205 is disposed within the barrel 203 and can slide inwardly and outwardly of the barrel 203 per the manipulations of a user. The plunger 205 has a plunger head 206 that forms a seal. Accordingly, in ordinary use, the plunger head 206 begins at a position that is most proximal to the syringe tip 204, and then, as the user withdraws the plunger 205 outwardly of the barrel 203, a vacuum forms between the plunger head 206 and the syringe tip 204 to thereby, in the case of a thrombectomy, draw into the barrel 203 material from within the patient's body. By another approach, the inner diameter (ID) of the syringe tip 204 and the ID of the catheter 101 are closely matched. By another approach, the ID of the inner lumen of the syringe 203, the ID of the syringe tip 204 and the ID of the catheter are closely matched. “Closely matched” may vary somewhat given a specific application setting, but will nevertheless comprise only a small variation as regards the inner diameter of both components. Examples include, but are not limited to, no more than a 1% difference, a 2% difference, a 3% difference, a 4% difference, a 5% difference, or a 10% difference, or any value within the range of about 0.5% to about 15% difference.

In this example, a thrombectomy catheter 101 connects with the syringe 102 via an intervening (initially closed) valve 103. The connection to the thrombectomy catheter 101 can be made at the distal end of the thrombectomy catheter 101 (which is the end of the thrombectomy catheter 101 that remains distant from the thrombus during use). It will be understood that this reference to the distal end of the thrombectomy catheter 101 includes not only the literal end of the thrombectomy catheter 101 but also to a portion of the thrombectomy catheter 101 that is distal from the working end thereof. Accordingly, and by way of a few illustrative examples, that distal portion can comprise one tenth, one fifth, one fourth, or one third of the total length of the thrombectomy catheter 101 as desired.

The syringe 102 may comprise a non-locking syringe or a locking syringe as desired. For many application settings, a non-locking syringe may be preferred. In some applications, an aspiration pump may be preferred.

The thrombectomy kit 100 also includes thrombectomy instructions 105 that instruct a user regarding how to use components of the thrombectomy kit 100 to remove a thrombus without a precharged vacuum in accordance with the present teachings. These instructions 105 may comprise, for example, a sheet or card of paper, a paper-based medium (such as card stock or cardboard), a plastic sheet or card, or other medium of choice. The instructions 105 may also comprise, for example, a QR code that leads the clinician's device (such as a smartphone or tablet) to on-line instructions.

The thrombectomy kit 100 can further include, if desired, one or more mechanical thrombectomy components 104. Such a mechanical thrombectomy component can be configured to be disposed through a patient's blood-carrying vessel (via, for example, the thrombectomy catheter 101) to a thrombus that is to be removed from the patient. Such a mechanical thrombectomy component 104 may comprise, for example, a clot retriever such as an entrapment cage 201. A stent retriever is one example in the foregoing regards. Such a stent retriever is configured to expand and encapsulate or capture the thrombus and to help maintain the clot structural integrity during the aspiration and removal process. In some applications, the mechanical thrombectomy component may include a basket or strainer type component to capture any portion of the blood clot that may break off during the thrombectomy procedure.

So configured, these teachings will accommodate disposing at least one mechanical thrombectomy component 104 through the thrombectomy catheter 101 and manipulating the at least one mechanical thrombectomy component 104 to remove a part of the at least one thrombus from the patient via the at least one mechanical thrombectomy component 104. This activity can be accompanied by aspiration carried out via the syringe 102 as described herein (or via an aspiration pump (not shown)) before, during, and/or after the use of the mechanical thrombectomy component 104.

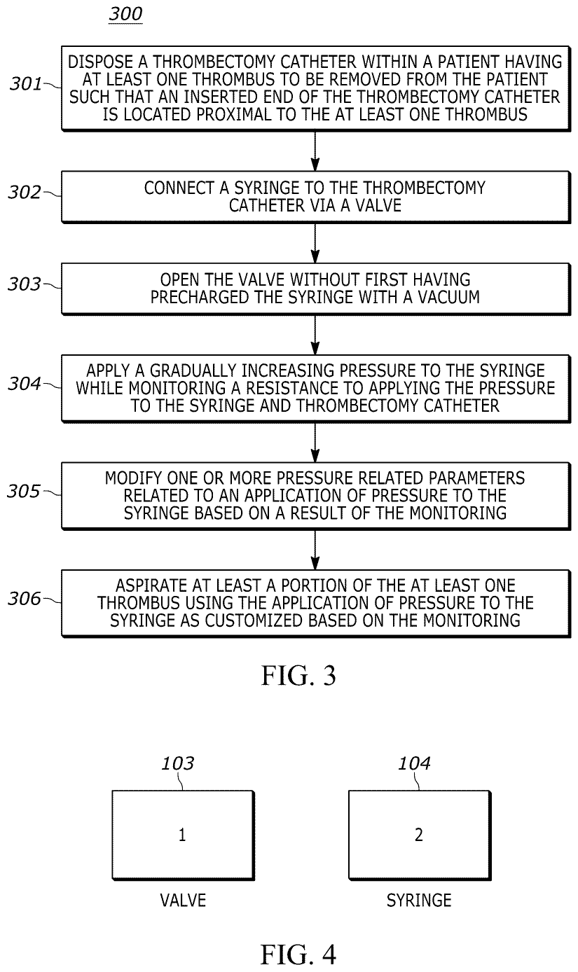

As shown in FIG. 1, these teachings will accommodate the thrombectomy kit optionally including a plurality of syringes 102, and as shown in FIG. 3, the thrombectomy kit may optionally also include a manifold 202 that is configured to operably couple the plurality of syringes 102 to the thrombectomy catheter 101. In this illustrative example, that manifold 202 is located on the same side of the valve 103 as the syringe 102. These additional syringes can be utilized, when and as needed, to aspirate a greater volume than what can be achieved with only a single syringe. In some examples, the manifold 202 and/or the valve 103 are replaced by a check valve or a slit valve (not shown), where the syringe 102 is coupled to the catheter via only the check valve or slit valve. In this case, the valve 103 can be automatically closed prior to the syringe connection and can become automatically open after the syringe is connected.

FIG. 3 presents a method 300 that can be carried out with the above-described thrombectomy kit 100. The aforementioned instructions 105 can include information conveying one or more of the following described steps.

Step 301 provides for disposing the thrombectomy catheter 101 within a patient having at least one thrombus to be removed from the patient such that an inserted end of the thrombectomy catheter 101 is located proximal to the at least one thrombus. Step 302 provides for connecting the syringe 102 to the thrombectomy catheter 101 via the valve 103. At this point in the method 300, the valve 103 is closed. Accordingly, the syringe 102 is not yet in fluid communication with the thrombectomy catheter 101.

It will also be observed that the syringe 102 is not precharged with a vacuum. By one approach, this reference to the syringe 102 not being precharged refers to the syringe plunger 205 being in a closed position, such that the plunger head 206 is most proximate to the syringe tip 204.

Step 303 provides for the clinician opening the valve 103 (again without first having precharged the syringe 102 with a vacuum) (as illustrated in FIG. 2 by the curved arrow denoted by the expression “1^{st}”) and then, at step 304, applying a gradually increasing pressure to the syringe 102 (for example, via the syringe's plunger as illustrated by the straight arrow in FIG. 2 denoted by the expression “2^{nd}”) while monitoring a resistance to applying the pressure to the syringe 102 and thrombectomy catheter 101. This monitoring is accomplished by the senses of the administering clinician themselves. For example, the amount of force required to pull the plunger outward of the syringe 102 can be felt by the clinician.

Step 305 provides for the clinician modifying one or more pressure-related parameters related to an application of pressure to the syringe 102 based on a result of the aforementioned monitoring. This one or more pressure-related parameters can comprise, for example, at least one of a speed of applying pressure, the force of applying pressure, a duration of applying pressure, and time intervals between each application of pressure. All of these can be self-determined by the clinician based, for example, on their experience and training, and implemented by use of their hands. This approach permits the clinician to tailor an aspiration operation to the characteristics of the thrombus (such as the size, location, and/or consistency of the thrombus) in a way that typical prior art approaches are unable to accommodate when using a pre-charged vacuum.

As a simple illustrative example, in a given procedure the clinician might withdraw the plunger by hand slowly a short distance and then stop. The plunger may then be withdrawn slowly again a similar short distance following a delay of a few seconds and then again stopped. This pattern may continue a few cycles, and then the clinician may pause for a longer duration of time, following which the clinician pushes the plunger back into the syringe 102 a short distance. After a short pause, the clinician may then again withdraw the plunger a short distance before again stopping. Variations on the foregoing can continue under the control of the clinician until the clinician is satisfied with the aspiration result. In some cases, based on the tactile or visual cues or feedback, the clinician may reverse the aspiration force applied to the plunger and push the plunger into the syringe cavity, reducing the pressure within the syringe cavity, if for example the clinician is anticipating that the application of immediate pressure on the blood clot may cause it to break apart. In some cases, the clinician may vary the pressure-related parameters based on the visual inspection of the content of aspirate within the syringe. For example, if it appears that the entire thrombosis has been aspirated into the syringe cavity, the clinician may reduce or stop the withdrawal of the syringe plunger.

These teachings additionally enable the clinician to adjust the applied suction force on the syringe 102 based on the visual clues of seeing the amount and consistency of the clot that is being collected in the syringe 102. This option is not available in systems where the application of the negative pressure created in a pre-charged syringe is applied all at once and almost instantly to the thrombectomy catheter 101, which allows little or no possibility of adjustment.

Step 306 provides for aspirating at least a portion of the at least one thrombus using the application of pressure to the syringe 102 as customized based on the monitoring described above.

If desired, at least some of the aforementioned components of the thrombectomy kit 100 can be supplemented with indicators to visually identify an order in which one or more of the components are to be used. For example, a visual indicator can serve to indicate that one component (such as the valve 103) should be used first and that a second component (such as the syringe 102) should be used second. By one approach, different colors can be used in such regards. Part or all of the valve 103 may be colored one color (such as green) while part or all of the syringe 102 (such as the plunger handle) may be colored a different color (such as red).

By another approach as illustrated in FIG. 4, in lieu of the foregoing or in combination therewith, alphanumeric characters and/or graphics can serve in these same regards. The valve 103, for example, may have the number “1” prominently displayed thereon and the syringe 102 may have the number “2” similarly displayed thereon to indicate an order in which these two user-manipulated components are to be used.

The aforementioned instructions 105 can include text and/or illustrations to explain the corresponding order-identification system. By another approach, the instructions 105 can include one or more illustrations that positively illustrate using the components of the thrombectomy kit 100 with a precharged vacuum and/or that negatively illustrate using the components of the thrombectomy kit 100 with a precharged vacuum.

If desired, and as illustrated in FIG. 1, the thrombectomy kit 100 can further optionally include mechanical linkage 106. By one approach this mechanical linkage 106 can mechanically couple the syringe 102 to the valve 103, such that withdrawing a plunger from the syringe 102 simultaneously opens the valve 103 to thereby prevent precharging a vacuum in the syringe 102. By another approach, this mechanical linkage 106 can be configured to prevent the plunger from being withdrawn from the syringe 102 unless and until the valve 103 is opened first.

By another approach, the valve 103 comprises a slit valve without a stopcock. The presence of the slit valve can prevent precharging of a vacuum in the syringe 102 when the syringe 102 is coupled to the catheter 101 via the slit valve. Slit valves are a specialized one-way valve that can prevent blood backflow while allowing passage of thrombus or embolic material. A slit valve typically comprises a flexible membrane (such as a rubber or rubber like material) having one or more slits formed therethrough that remain closed at rest, thus maintaining a fluid-tight seal. The slit valve allows for the insertion of the syringe tip 204 into the valve slit, forming the fluid-tight seal around the syringe tip 204, preventing any fluids (such as blood) from oozing out. Upon concluding the withdrawal of the syringe barrel 203 to aspirate all or part of the thrombus, the slit(s) remain closed and maintain the fluid-tight seal around the syringe tip 204. Based on the properties of the slit valve flexible membrane, the slit valve may tolerate a maximum amount of pressure while maintaining a fluid-tight seal. Exceeding the maximum level of pressure, the slit valve may lose its ability to maintain the fluid-tight seal and leak, thus preventing the application of excessive pressure to the thrombus, causing the thrombus to break apart. By one approach, if desired, such a slit valve may be incorporated into the tip 204 of the syringe 102, thus obviating a need for an external valve.

So configured, these teachings can provide increased and constantly available direct feedback and control to the clinician to facilitate careful and appropriate modulation of the suction force. This approach can result in an improved process, reduced clot breakup, and an improved outcome for the patient.

Further aspects of these teachings are provided by the subject matter of the following clauses (where it will be understood that any of these clauses can be combined with any one of more of the other clauses as desired).

Clause 1. A thrombectomy kit comprising: a thrombectomy catheter having a distal end; at least one syringe; a valve configured to operably couple between the syringe and the distal end of the thrombectomy catheter; thrombectomy instructions instructing a user to use components of the thrombectomy kit to remove a thrombus without a precharged vacuum.

Clause 2. The thrombectomy kit of clause 1 wherein the valve and the syringe include indicators to visually identify which is to be used first when initiating removal of a thrombus.

Clause 3. The thrombectomy kit of clause 2 wherein the indicators comprise different colors.

Clause 4. The thrombectomy kit of clause 2 wherein the indicators comprise graphics.

Clause 5. The thrombectomy kit of clause 2 wherein the indicators comprise alphanumeric characters.

Clause 6. The thrombectomy kit of clause 1 further comprising: mechanical linkage that couples the syringe to the valve, such that withdrawing a plunger from the syringe simultaneously opens the valve to thereby prevent precharging a vacuum.

Clause 7. The thrombectomy kit of clause 1 wherein the at least one syringe comprises a plurality of syringes and wherein the thrombectomy kit further comprises: a manifold configured to operably couple the plurality of syringes to the thrombectomy catheter via the valve.

Clause 8. The thrombectomy kit of clause 1 wherein the thrombectomy instructions include illustrations that positively illustrate using the components of the thrombectomy kit without a precharged vacuum.

Clause 9. The thrombectomy kit of clause 8 wherein the thrombectomy instructions include illustrations that negatively illustrate using the components of the thrombectomy kit with a precharged vacuum.

Clause 10. The thrombectomy kit of clause 1 further comprising: at least one mechanical thrombectomy component configured to be disposed through a patient's blood-carrying vessel to a thrombus that is to be removed from the patient.

Clause 11. The thrombectomy kit of clause 10 wherein the at least one mechanical thrombectomy component comprises a clot retriever.

Clause 12. The thrombectomy kit of clause 11 wherein the clot retriever comprises a clot entrapment cage.

Clause 13. A thrombectomy apparatus comprising: a valve configured for use with a thrombectomy catheter; a syringe configured to aspirate a thrombus; wherein the valve and the syringe include indicators to visually identify which is to be used first when initiating removal of a thrombus without use of a precharged vacuum.

Clause 14. The thrombectomy apparatus of clause 13 wherein the indicators comprise different colors.

Clause 15. The thrombectomy apparatus of clause 13 wherein the indicators comprise graphics.

Clause 16. The thrombectomy apparatus of clause 13 wherein the indicators comprise alphanumeric characters.

Clause 17. The thrombectomy apparatus of clause 13 further comprising: mechanical linkage that couples the syringe to the valve, such that withdrawing a plunger from the syringe simultaneously opens the valve to thereby prevent precharging a vacuum.

Clause 18. A thrombectomy method comprising: disposing a thrombectomy catheter within a patient having at least one thrombus to be removed from the patient such that an inserted end of the thrombectomy catheter is located proximal to the at least one thrombus; connecting a syringe to the thrombectomy catheter via a closed valve; opening the valve without first having precharged the syringe with a vacuum; applying a gradually increasing pressure to the syringe while monitoring a resistance to applying the pressure to the syringe and thrombectomy catheter; modifying one or more pressure-related parameters related to an application of pressure to the syringe based on a result of the monitoring; aspirating at least a portion of the at least one thrombus using the application of pressure to the syringe as customized based on the monitoring.

Clause 19. The thrombectomy method of clause 18 wherein the one or more pressure-related parameters comprise at least one of a speed of applying pressure, a duration of applying pressure, and time intervals between each application of pressure.

Those skilled in the art will recognize that a wide variety of modifications, alterations, and combinations can be made with respect to the above-described embodiments without departing from the scope of the invention, and that such modifications, alterations, and combinations are to be viewed as being within the ambit of the inventive concept.

1. A thrombectomy kit comprising:
a thrombectomy catheter having a distal end;
at least one syringe;
a valve configured to operably couple between the syringe and the distal end of the thrombectomy catheter;
thrombectomy instructions instructing a user to use components of the thrombectomy kit to remove a thrombus without a precharged vacuum.

2. The thrombectomy kit of claim 1 wherein the valve and the syringe include indicators to visually identify which is to be used first when initiating removal of a thrombus.

3. The thrombectomy kit of claim 2 wherein the indicators comprise different colors.

4. The thrombectomy kit of claim 2 wherein the indicators comprise graphics.

5. The thrombectomy kit of claim 2 wherein the indicators comprise alphanumeric characters.

6. The thrombectomy kit of claim 1 further comprising:
mechanical linkage that couples the syringe to the valve, such that withdrawing a plunger from the syringe simultaneously opens the valve to thereby prevent precharging a vacuum.

7. The thrombectomy kit of claim 1 wherein the at least one syringe comprises a plurality of syringes and wherein the thrombectomy kit further comprises:
a manifold configured to operably couple the plurality of syringes to the thrombectomy catheter via the valve.

8. The thrombectomy kit of claim 1 wherein the thrombectomy instructions include illustrations that positively illustrate using the components of the thrombectomy kit without a precharged vacuum.

9. The thrombectomy kit of claim 1 wherein the valve comprises a slit valve.

10. The thrombectomy kit of claim 1 further comprising: at least one mechanical thrombectomy component configured to be disposed through a patient's blood-carrying vessel to a thrombus that is to be removed from the patient.

11. The thrombectomy kit of claim 10 wherein the at least one mechanical thrombectomy component comprises a clot retriever.

12. The thrombectomy kit of claim 11 wherein the clot retriever comprises a clot entrapment cage.

13. A thrombectomy apparatus comprising:
a valve configured for use with a thrombectomy catheter;
a syringe configured to aspirate a thrombus;
wherein the valve and the syringe include indicators to visually identify which is to be used first when initiating removal of a thrombus without use of a precharged vacuum.

14. The thrombectomy apparatus of claim 13 wherein the indicators comprise different colors.

15. The thrombectomy apparatus of claim 13 wherein the indicators comprise graphics.

16. The thrombectomy apparatus of claim 13 wherein the indicators comprise alphanumeric characters.

17. The thrombectomy apparatus of claim 13 further comprising:
mechanical linkage that couples the syringe to the valve, such that withdrawing a plunger from the syringe simultaneously opens the valve to thereby prevent precharging a vacuum.

18. A thrombectomy method comprising:
disposing a thrombectomy catheter within a patient having at least one thrombus to be removed from the patient such that an inserted end of the thrombectomy catheter is located proximal to the at least one thrombus;
connecting a syringe to the thrombectomy catheter via a closed valve;
opening the valve without first having precharged the syringe with a vacuum;
applying a gradually increasing pressure to the syringe while monitoring a resistance to applying the pressure to the syringe and thrombectomy catheter;
modifying one or more pressure-related parameters related to an application of pressure to the syringe based on a result of the monitoring;
aspirating at least a portion of the at least one thrombus using the application of pressure to the syringe as customized based on the monitoring.

19. The thrombectomy method of claim 18 wherein the one or more pressure-related parameters comprise at least one of a speed of applying pressure, a duration of applying pressure, and time intervals between each application of pressure.
